(19) United States
(12) Patent Application Publication
(10) Pub. No.: US 2017/0231261 A1
(43) Pub. Date: Aug. 17, 2017

(54) NUTRITIONAL COMPOSITION WITH LOW CONTENT OF MEDIUM-CHAIN FATTY ACIDS IN SPECIFIC PROPORTIONS, AND ITS USES

(71) Applicant: NESTEC S.A., Vevey (CH)

(72) Inventors: Sagar Thakkar, Brent (CH); Kornel Nagy, Lausanne (CH); Clara Lucia Garcia-Rodenas, Forel (CH); Kim-Anne Le, Lausanne (CH); Timothy James Wooster, Epalinges (CH)

(73) Assignee: NESTEC S.A., Vevey (CH)

(21) Appl. No.: 15/515,347

(22) PCT Filed: Sep. 29, 2015
(86) PCT No.: PCT/EP2015/072391

§ 371 Date: Mar. 29, 2017

(30) Foreign Application Priority Data
Sep. 30, 2014 (EP) 14187057.6

(51) Int. Cl. A23L 33/00; A23L 33/12; A23L 33/17; A61K 31/20; A61K 9/00; A61K 36/889; A61K 31/201; A61K 31/202; A61K 36/28; A61K 36/31; A23L 33/115; A61K 31/19

(52) U.S. Cl. CPC A23L 33/40; A23L 33/115; A23L 33/12; A23L 33/17; A61K 31/20; A61K 31/19; A61K 36/889; A61K 31/201; A61K 31/202; A61K 36/28; A61K 36/31; A61K 9/0095; A23V 2200/30

(57) ABSTRACT

The present invention relates to synthetic nutritional compositions, especially infant formula compositions, with a low content of medium-chain fatty acids (or MFCAs) and particularly in specific proportions. The invention also relates to the use of said compositions to promote liver and/or gut maturation in infants or young children and/or to reduce the risk of liver and/or gut disease in infants or young children.

### TECHNICAL FIELD OF THE INVENTION

The present invention relates to synthetic nutritional compositions, especially infant formula compositions, and in particular synthetic nutritional compositions suitable for nutrition of infants and/or young children with low birth weight (LBW), who were born preterm, who experienced intra-uterine growth retardation, who were small for gestational age, and/or who are critically ill. The synthetic nutritional compositions of the invention have a low content of medium-chain fatty acids (or MCFAs) in specific proportions.

The invention also relates to the synthetic nutritional compositions of the invention for use to promote liver and/or gut maturation, and/or to reduce the risk of liver and/or gut disease, in infants and/or young children, in particular in infants and/or young children with low birth weight (LBW), who were born preterm, experienced intra-uterine growth retardation, who were small for gestational age, and/or who are critically ill.

### BACKGROUND OF THE INVENTION

A well-nourished mother's breast milk is universally considered to be the optimum nutrition for healthy full term infants during the first months of life. However all infants cannot be breast-fed. Furthermore, the needs of more vulnerable infants such as infants or young children low birth weight (LBW), who were born preterm, who experienced intra-uterine growth retardation, who were small for gestational age, who are critically ill and/or who suffered from growth delays due to disease and/or malnutrition cannot be achieved by their mother's milk. Synthetic nutritional compositions, in particular infant formulas are therefore of high interest.

Lipids comprise an important part of synthetic nutritional compositions because they provide a substantial part of the energy content, they are a source of n-3 and n-6 essential fatty acids and they are necessary for the intestinal absorption of fat-soluble vitamins.

The efficiency of intestinal absorption of fatty acids is an important property of the fat blend. As a general principle, it is believed that shorter-chain fatty acids are better absorbed than longer chain fatty acids, and unsaturated fatty acids are better absorbed than saturated fatty acids of the same chain length.

In order to achieve good fat absorption, synthetic nutritional composition suitable for infants and/or young children with LBW, who were born preterm and/or who experienced intra-uterine growth retardation and/or who are critically ill usually contain a high proportion of MCT oil.

MCT oils are a food ingredient product. They are usually manufactured by refining vegetable oils rich in medium chain fatty acids (MCFAs), and are widely used in synthetic nutritional compositions including infant formulas, especially infant formulas intended for vulnerable infants and young children.

In regards of infant formulas suitable for preterm feeding, recommendations are set out by European Society for Paediatric Gastroenterology, Hepatology and Nutrition (ESPGHAN) 2010 and LSRO 2002. According to the ESPGHAN products shall contain <40% Medium chain triacylglycerols; and in accordance to LSRO: ≦50% MCT oil.

U.S. Pat. No. 5,000,975 describes infant formula compositions in which the fat is issued from vegetable oil fat compositions. For preterm and low birth weight infants, MCTs are included in the composition in a total amount of 10 to 25 wt % with respect to the total fat. These MCTs are made up of a mixture of C6:0 (1 to 2%), C8:0 (65 to 75%), C10:0 (25 to 35%) and C12:0 (1 to 2%) fatty acids, derived from coconut oil. These MCTs comprise predominantly C8:0 and C10:0 fatty acids, in amounts of 60-70% of octanoic acid (C8:0) and of 25-35% of decanoic acid (C10:0).

More recently, U.S. Pat. No. 5,709,888 describes fat mixtures, in particular for infant nutrition, comprising between about 4.8 to about 28.7% by weight of MCTs in total, with respect to the total fat. In some examples where the total amount of MCTs is of 7.85 to 8.0 wt %, with respect to the total fat, the amount of octanoic acid (C8:0) is around 2.35 to 2.50 wt %, the amount of decanoic acid (C10:0) is of 3.60 to 3.80 wt % and the amount of dodecanoic acid (C12:0) is of 1.80 to 2.00 wt %, with respect to the total fat.

However MCFA-rich meals can increase the risk of hepatic disease, in particular the risk of hepatic steatosis (Turner, 2009). Also the digestive function is poorly stimulated by MCFA-rich meals. Intagastric lipase content is higher in preterms fed with LCFAs-rich formula than with MCFAs formula (Hamosh, 1991). In addition, contrary to LCFA, MCFA-rich meals do not stimulate CCK, pancreatic exocrine and bile secretion (Vu, 1999).

Thus there is a need to provide a nutritional system that enables the convenient, safe and accurate delivery of the most adequate nutrition to infants and/or young children, in particular to infants and/or young children with LBW, who were born preterm and/or who experienced intra-uterine growth retardation and/or who are critically ill.

There is furthermore a need to provide nutritional solutions which promote liver and/or gut maturation in infants and/or young children, reduce the risk of liver and/or gut disease in infants and/or young children, in particular vulnerable infants and/or young children, such as infants and/or young children with LBW, who are critically ill, who were born preterm, who experienced intra-uterine growth retardation and/or who suffered from growth delays due to disease and/or malnutrition.

### SUMMARY OF THE INVENTION

The present inventors have found surprisingly that the synthetic nutritional compositions according to the invention are emptied more rapidly from the stomach, despite having a high content of LCFAs relative to MCFAs. Thus, the synthetic nutritional compositions of the present invention make it possible to provide the benefits of LCFAs without slowing gastric emptying. Furthermore, the compositions according to the invention display increased stimulation of gut digestive capacity as well as of gut hormones [e.g. Fibroblast growth factor 19 (FGF-19) and cholecystokinin (CCK)].

Accordingly, in a first aspect, the invention relates to a synthetic nutritional composition comprising
medium-chain fatty acids, mainly in the form of triglycerides, in a total amount in the range of 5 to 20 wt % with respect to the total fat,
hexanoic acid in an amount in the range of 0 to 0.5 wt % with respect with the total fat;
octanoic acid in an amount in the range of 1.55 to 2 wt % with respect with the total fat;
decanoic acid in an amount in the range of 0 to 1.8 wt % with respect with the total fat;
dodecanoic acid in an amount in the range of 0 to 12.0 wt % with respect with the total fat, and
at least one long chain fatty acid, mainly in the form of triglycerides, in an amount of at least 15 wt % with respect to the total fat.

In one embodiment, the invention relates to a synthetic nutritional composition comprising
medium-chain fatty acids, mainly in the form of triglycerides, in a total amount in the range of 5 to 20 wt % with respect to the total fat,
hexanoic acid in an amount in the range of 0.05 to 0.5 wt % with respect with the total fat;
octanoic acid in an amount in the range of 1.55 to 2 wt % with respect with the total fat;
decanoic acid in an amount in the range of 0 to 1.8 wt % with respect with the total fat;
dodecanoic acid in an amount in the range of 0 to 12.0 wt % with respect with the total fat, and
at least one long chain fatty acid, mainly in the form of triglycerides, in an amount of at least 15 wt % with respect to the total fat.

In one embodiment of this aspect, the synthetic nutritional composition is a fat blend.

Another aspect of the present invention relates to the use of a synthetic nutritional composition according to the invention, for example when the synthetic nutritional composition is a fat blend, for the preparation of a synthetic nutritional composition according to the invention, for example an infant formula.

A further aspect of the present invention relates to the synthetic nutritional compositions for use in promoting and/or ensuring gut maturation, reducing the risk of gut atrophy; increasing gastric emptying rate; reducing the accumulation of gastric residuals; improving tolerance to feeds in an infant or young child; promoting and/or ensuring liver maturation and/or reducing the risk of liver disease in particular in a vulnerable infant or young child, such as infants and/or young children with LBW, and/or who are critically ill, and/or who were born preterm, and/or who experienced intra-uterine growth retardation and/or who suffered from growth delays due to disease and/or malnutrition, and/or who have been fed enterally or are at risk for becoming candidates for enteral feeding.

Another embodiment of this aspect relates to the synthetic nutritional compositions according to the invention for use in promoting optimal growth and reducing the risk of diabetes, obesity and/or metabolic syndrome, such as for use in use in promoting insulin sensitivity, improving glucose management, improving nutrient utilization, improving lean body mass growth, reducing visceral adiposity and/or reducing the risk of metabolic disease later in life in an infant or a young child.

Finally, the invention in a yet further aspect relates to a method of treating and/or preventing liver disease, diabetes, obesity and/or metabolic syndrome, comprising administering a synthetic nutritional composition according to the invention to an individual in need thereof, for example an infant, young child, child or adult, especially to a vulnerable infant or young child.

### BRIEF DESCRIPTION OF THE FIGURES

FIG. 1 show an overview of the study described in Example 1.

FIG. 2 show the results for measurements of gastric emptying in Example 1. Dots represent the estimated means and the bars the 95% confident interval of the half gastric emptying times after consumption of the different meals. M=Meal (see Table 1 and Tables 2 to 6 for composition); * indicates P<0.0001; ** indicates P=0.0003. Half time (t50/min)

FIG. 3 and FIG. 5 show the results for measurements of FGF 19 in blood samples taken during the experiments described in Example 1. In FIG. 3, dots represent the estimated means and the bars the 95% confident interval of the area under the curve of the FGF 19 secretion kinetics, corrected by the FGF19 baseline values. M=Meal (see Table 1 and Tables 2 to 6 for composition); ** indicates P=0.001. Area over baseline (AOB), (picogram/mL min). FIG. 5 is a box plot of the FGF19 maximum levels (picogram/mL) after correction by the FGF19 baseline values. * indicates meals where Mx>M1, p<0.05

FIG. 4 and FIG. 6 show the results for measurement of bile acids in blood samples taken during experiment described in Example 1. In FIG. 4 dots represent the estimated means and the bars the 95% confident interval of the area under the curve of the bile acid secretion kinetics, corrected by the bile acid baseline values. Area over baseline (AOB), (micromol/L min). * indicates P=0.9; ** indicates P=0.3; *** indicates P=0.0003; **** indicated P=0.0001. FIG. 6 is a box plot of the bile acid maximum levels (micromol/L) after correction by the bile acid baseline values. * indicates meals where Mx>M1, p<0.05.

FIGS. 7 and 8 shows the results for measurement of cholecystokinin (CCK) in blood samples taken during the experiment described in Example 1 FIG. 7 is a box plot of the area under the curve of CCK secretion kinetics, corrected by the bile acid baseline values (picomol/L min) after correction by the CCK baseline values. * indicates meals where Mx>M1, p<0.05. FIG. 8 is a box plot of the CCK maximum levels (picomol/L) after correction by the CCK baseline values. * indicates meals where Mx>M1, p<0.05; # indicates meals where Mx<M1, p<0.05.

### DETAILED DESCRIPTION OF THE INVENTION

### Definitions

Prior to discussing the present invention in further details, the following terms and conventions will first be defined:

The term “synthetic” means obtained by chemical and/or biological means, to the contrary of “natural (found in the nature)”, for example not of human origin (e.g. this is not breast milk). Even though the components may be natural, the composition as described is not found in nature.

The term “nutritional” means that it nourishes a subject. The synthetic nutritional compositions according to the invention are typically to be taken by enteral feeding, such as by intragastric (e.g. oro-gastric, nasogastric), intraduodenal or intrajejunal, feeding, and may include a carbohydrate and/or a protein source. The synthetic nutritional compositions according to the invention may be intended for infants and/or young children, and may be for example be formulated as an infant formula, growing up-milk, any other milk-based nutritional composition, a supplement (or a complement), a fortifier such as a milk fortifier (e.g. human milk fortifier). The nutritional compositions can be in powder or liquid form.

The expression “infant formula” means a foodstuff intended for particular nutritional use of infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). It has to be understood that infants can be fed solely with infant formulas, or that the infant formula can be used as a complement of human milk.

The infant formulas can encompass the starter infant formulas and the follow-up or follow-on formulas. Generally a starter formula is for infants from birth as breast-milk substitute. A follow-up or follow-on formula is typically given from the 6th month onwards. It constitutes the principal liquid element in the progressively diversified diet of this category of person.

The expression “preterm infant formula” means an infant formula intended for a preterm infant.

The term “growing-up milk” are given from one year onwards. It is generally a milk-based beverage adapted for the specific nutritional needs of young children.

The term “fortifier” means a nutritional composition for infants intended to be added to or diluted with an infant formula or with human milk.

The term “supplement” means a nutritional composition which typically provides the selected nutrients while not representing a significant portion of the overall nutritional needs of the subject. Typically they do not represent more than 0.1%, 1%, 5%, 10% or 20% of the daily energy need of the subject. Supplements can for example be provided in the form of a pill, a tablet or a powder supplement that can for example be dissolved in water or sprinkled on food. Most preferred is a powder supplement that can be dissolved in liquid or sprinkled on food, most preferably dissolved in water.

The term “complement” means the same as supplement, and the terms are used interchangeably herein. The synthetic nutritional composition according to the invention may be a hypoallergenic nutritional composition. The expression “hypoallergenic nutritional composition” means a nutritional composition which is unlikely to cause allergic reactions.

According to the Commission Directive 2006/141/EC of 22 Dec. 2006 on infant formulae and follow-on formulae, articles 2 (a) and (b), the term “infant” means a child under the age of 12 months. The expression “young child” means a child aged between one and up to three years of age, also called toddler.

The term “preterm” or “premature” means an infant or young child who was not born at term. Generally, it refers to an infant or young child who was born at less than 37 weeks gestational age. The expressions “infant(s)/young child(ren) born preterm”, “infant(s)/young child(ren) who is/are born preterm”, “preterm infant(s)/young child(ren)” and “preterms” can be used interchangeably.

A “term infant/young child” refers to an infant or young child born at term. Generally, it refers to an infant or young child who was born at 37 weeks of gestation or after.

By the expression “small for gestational age” or “SGA”, it is intended to mean an infant or young child who is smaller in size than normal for their gestational age at birth, most commonly defined as a weight below the 10^{th} percentile for the gestational age. In some embodiments, SGA may be associated with Intrauterine growth restriction (IUGR), which refers to a condition in which a fetus is unable to achieve its potential size.

By the expression “low birth weight”, it should be understood as any body weight under 2500 g at birth. It therefore encompasses:
infant or young child who has/had a body weight from 1800 to 2500 g at birth (usually called “low birth weight” or LBW)
infant or young child who has/had a body weight from 1000 to 1800 g at birth (called “very low birth weight” or VLBW)
infant or young child who has/had a body weight under 1000 g at birth (called “extremely low birth weight” or ELBW)

Infants or young children with low birth weight may or may not be preterm, and similarly, infants or young children who were small for gestational age may or may not be preterm.

The term “vulnerable infants and/or young children” is used in the present application to refer to infants and/or young children who have or have had development challenges, such as infants and/or young children with at least one of the following features: with LBW, who are critically ill, who were born preterm, who were small for gestational age, who experienced intra-uterine growth retardation and/or who suffer(ed) from growth delays due to disease and/or malnutrition.

The term “critically ill” refers to individuals with a life threatening illness or injury. Such individuals, for example an infant or young child, may require enteral, oro-gastric, nasogastric, intragastric, intraduodena or intrajejunal feeding, and may be at risk for parenteral feeding.

The expression “medium-chain fatty acid” (or MCFA) refers to n-carboxylic acids of saturated linear aliphatic chains, and are in total comprised of 6-12 carbon atoms. The medium-chain fatty acids of the present invention may be hexanoic acid (comprising 6 carbon atoms, C6:0 also known as caproic acid), octanoic acid (comprising 8 carbon atoms, C8:0, also known as caprylic acid), decanoic acid (comprising 10 carbon atoms or C10:0, also known as capric acid) and/or dodecanoic acid (comprising 12 carbon atoms or C12:0, also known as lauric acid).

The expression “gastric residuals” refers to food from a previous feeding left in the stomach at the start of the next feeding.

In the synthetic nutritional composition of the invention, the medium-chain fatty acids are present mainly (i.e. at least 98%, such as more than 98.5%, or more than 99% or more than 99.5%) in the form of triglycerides. The term “medium-chain triglycerides” (or MCT) means medium-chain fatty acid esters of glycerol, that is to say a compound formed of a glycerol backbone and three fatty acids. In the case of MCTs, two or three of the fatty acid chains attached to glycerol are medium chain in length. In the context of the present application, the terms “triglyceride”, “triacylglyceride” and “triacylglycerol” are used interchangeably and mean the same compound.

The term “MCT oil” refers to a manufactured product comprising medium chain triglycerides.

The expression “long-chain fatty acid” (or LCFA) refers to n-carboxylic acids of saturated or (poly)unsaturated branched or linear aliphatic chains, and are in total comprised of greater than 12 carbon atoms. Examples of long chain fatty acids include palmitic acid (comprising 16 carbon atoms, or C16:0) and Oleic acid (18:1 cis-9). Similarly, the long-chain fatty acids of the present invention are mainly (i.e. at least 98%, such as more than 98.5%, or more than 99% or more than 99.5%) in the form of triglycerides. The expression “long chain triglycerides” (or LCT) means long chain fatty acid esters of glycerol, that is to say a compound formed of a glycerol backbone and three fatty acids, two or three fatty acid chains attached to glycerol being long chain in length.

The expression “long-chain polyunsaturated fatty acid” (or LC-PUFA) means a polyunsaturated fatty acid (PUFA) having C20 or C22 carbon atoms. Polyunsaturated fatty acids (PUFAs) are unsaturated fatty acids that contain more than one double bond in their backbone.

By the expression “palmitic acid sn 1,3”, it is intended to mean palmitic acid esterified in the sn 1,3 position.

By the expression “palmitic acid sn 2”; it is intended to mean palmitic acid esterified in the sn 2 position.

The term “wt %” means percentage by weight. All percentages are by weight of total fat unless otherwise stated.

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “probiotic” means microbial cell preparations or components of microbial cells with a beneficial effect on the health or well-being of the host (Salminen S, Ouwehand A. Benno Y. et al. “Probiotics: how should they be defined” Trends Food Sci. Technol. 1999:10 107-10).

The expression “consists essentially of” means that in addition to those components which are mandatory, other components may also be present in the composition, provided that the essential characteristics of the composition are not materially affected by their presence.

As used in this specification, the words “comprises”, “comprising”, and similar words, are not to be interpreted in an exclusive or exhaustive sense. In other words, they are intended to mean “including, but not limited to”.

It should be noted that embodiments and features described in the context of one of the aspects of the present invention also apply to the other aspects of the invention.

All patent and non-patent references cited in the present application, are hereby incorporated by reference in their entirety.

Any reference to prior art documents in this specification is not to be considered an admission that such prior art is widely known or forms part of the common general knowledge in the field.

### Synthetic Nutritional Composition

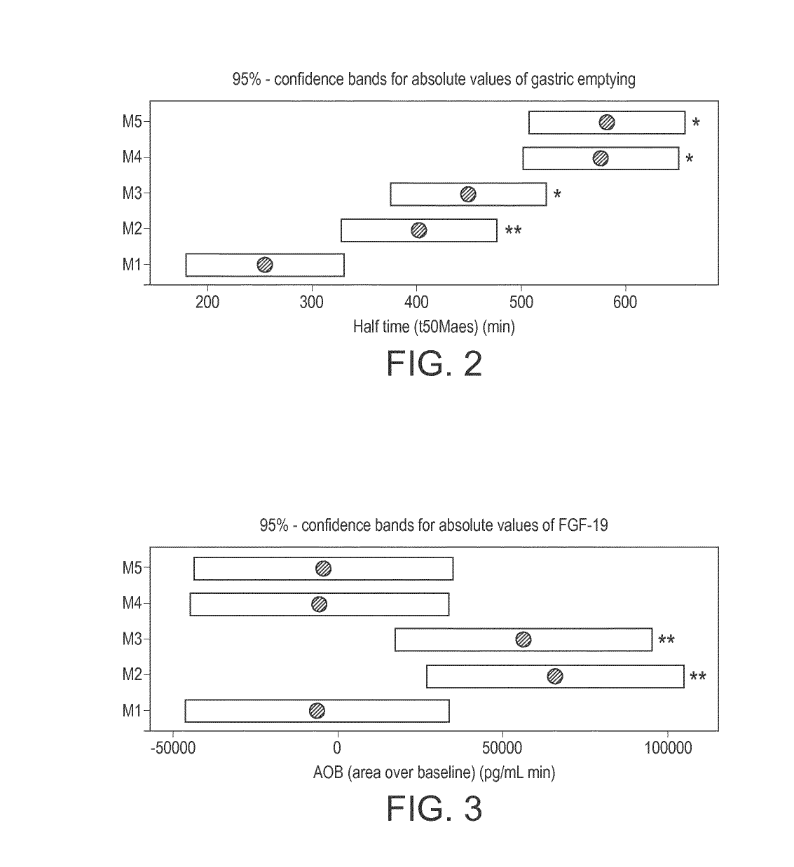

The nutritional compositions of the invention show increased gastric emptying rate, despite having high amounts of LCFAs (see results in Example 1, and FIG. 2).

Additionally, LCFAs have many reported benefits which are also surprisingly achieved by the composition of the invention. Intragastric lipase content is higher in preterms fed with LCFAs-rich formula than with MCFAs formula (Hamosh, 1991). In addition, contrary to LCFA, MCFA-rich meals do not stimulate cholecystokinin (CCK), pancreatic exocrine and bile secretions (Vu, 1999). Furthermore, the superior mesenteric artery (SMA) blood flow is more stimulated with LCFAs than with MCFAs (Vu, 2001). Impaired SAM blood flow is associated with intolerance to enteral feeds in preterms (Fang, 2001; Robel-Tillig, 2004).

However, LCFAs have also been associated with longer gastric emptying times.

Some infants suffer from immature gastrointestinal motility of the gut system which leads to slow transit, delayed gastric emptying, and accumulation of the bolus in the stomach (i.e. gastric residuals). This is especially the case vulnerable infants and/or young children, for example preterm infants.

Thus, it appeared that nutritional compositions based on LCFAs would be contraindicated, based on the assumption that LCFAs would further reduce motility, and/or further increase accumulation of gastric residuals. Indeed, MCTs are widely used in conventional nutritional formulae for infants in order to avoid these issues.

The present invention is based on the surprising finding that certain nutritional compositions comprising relatively high amounts of LCFAs mainly in the form of LCT, actually increase rate of gastric emptying. The compositions of the invention therefore provide a way of supplying the nutritional benefits of LCFAs, even in individuals where the gut is compromised, for example in vulnerable infants and/or young children, such as an infant and/or young child with immature gut. The absorption of LCFAs was accompanied by an increase in the gut digestive capacity (as reflected by increased bile salt secretion) as well as of gut hormones (i.e. cholecystokinin (CCK)). The increased absorption of LCFAs was also accompanied by an increase in Fibroblast Growth Factor 19 (FGF-19).

FGF 19 is an intestinal hormone that can reduce the risk of hepatic steatosis and cholestasis, and promote liver growth. Intestinal FGF-19 can also exert beneficial effects on glucose metabolism by increasing insulin sensitivity and inducing glycogen synthesis. Furthermore, FGF-19 can also promote protein synthesis, and lead to growth of lean body mass (Schaap, 2012; Reue, 2014).

Thus, the compositions of the invention are useful also in promoting balanced growth, and to treat and/or prevent obesity, metabolic syndrome (including improving insulin sensitivity and glucose tolerance), and/or diabetes.

### Fatty Acids—MCFAs

The invention in a first aspect relates to a synthetic nutritional composition comprising
medium-chain fatty acids, mainly in the form of triglycerides, in a total amount in the range of 5 to 20 wt % with respect to the total fat,
hexanoic acid in an amount in the range of 0 to 0.5 wt % with respect with the total fat;
octanoic acid in an amount in the range of 1.55 to 2 wt % with respect with the total fat;
decanoic acid is an amount in the range of 0 to 1.8 wt % with respect with the total fat;
dodecanoic acid in an amount in the range of 0 to 12.0 wt % with respect with the total fat, and
at least one long chain fatty acid, mainly in the form of triglycerides in an amount of at least 15 wt % with respect to the total fat.

One embodiment of this aspect relates to to a synthetic nutritional composition comprising:
medium-chain fatty acids, mainly in the form of triglycerides, in a total amount in the range of 5 to 20 wt % with respect to the total fat,
hexanoic acid in an amount in the range of 0.05 to 0.5 wt % with respect with the total fat;
octanoic acid in an amount in the range of 1.55 to 2 wt % with respect with the total fat;
decanoic acid is an amount in the range of 0 to 1.8 wt % with respect with the total fat;
dodecanoic acid in an amount in the range of 0 to 12.0 wt % with respect with the total fat, and
at least one long chain fatty acid, mainly in the form of triglycerides in an amount of at least 15 wt % with respect to the total fat.

One embodiment of this aspect relates to a synthetic nutritional composition according to the invention comprising medium-chain fatty acids mainly in the form of triglycerides, in a total amount in the range of 10.1 to 15 wt %, with respect to the total fat, wherein:
hexanoic acid is in an amount in the range of 0 to 0.5 wt % with respect with the total fat;
octanoic acid is in an amount in the range of 1.55 to 2 wt % with respect with the total fat;
decanoic acid is in an amount in the range of 1 to 1.8 wt % with respect with the total fat; and
dodecanoic acid is in an amount in the range of 8.0 to 12.0 wt % with respect with the total fat, and at least one long chain fatty acid, mainly in the form of triglycerides, is in an amount of at least 15 wt % with respect to the total fat.

Another embodiment of this aspect relates to a synthetic nutritional composition according to the invention comprising medium-chain fatty acids mainly in the form of triglycerides, in a total amount in the range of 10.1 to 15 wt %, with respect to the total fat, wherein:
hexanoic acid is in an amount in the range of 0.05 to 0.5 wt % with respect with the total fat;
octanoic acid is in an amount in the range of 1.55 to 2 wt % with respect with the total fat;
decanoic acid is in an amount in the range of 1 to 1.8 wt % with respect with the total fat; and
dodecanoic acid is in an amount in the range of 8.0 to 12.0 wt % with respect with the total fat, and at least one long chain fatty acid, mainly in the form of triglycerides, is in an amount of at least 15 wt % with respect to the total fat.

Another embodiment of this aspect relates to a synthetic nutritional composition comprising medium-chain fatty acids mainly in the form of triglycerides, in a total amount in the range of 5 to 20 wt % with respect to the total fat, and long chain fatty acids mainly in the form of triglycerides, in a total amount in the range of 85 to 90 wt % with respect to the total fat, and wherein the composition comprises palmitic acid sn 1,3 in an amount of no more than 10 wt % with respect to total fat.

In some embodiments of the invention, the synthetic nutritional composition comprises medium-chain fatty acids mainly in the form of triglycerides in a total amount in the range of 10 to 15 wt %, with respect to total fat; such as from 10.1 to 15%, from 10.5 to 15%, from 11 to 15%, from 12 to 15%, from 13 to 15%, from 14 to 15%, or for example from 10.1 to 14%, from 10.5 to 14%, or from 10.5 to 13%, or from 11 to 15%, or from 12 to 15%, all percentages by wt.

Further embodiments relates to a synthetic nutritional composition according to the invention, and wherein:
hexanoic acid is in an amount in the range of 0.05 to 0.5 wt % with respect with the total fat; such as for example 0.05 to 0.4, 0.06 to 0.4, or 0.07 to 0.3 wt %;
octanoic acid is in an amount in the range of 1.55 to 2 wt % with respect with the total fat; such as for example 1.6 to 2, 1.65 to 2, 1.7 to 2 wt %;
decanoic acid is in an amount in the range of 0 to 1.8 wt % with respect with the total fat; such as for example 1 to 1.8, 1 to 1.7, 1 to 1.5 or 1.2 to 1.8 wt %; and
dodecanoic acid is in an amount in the range of 0 to 12.0 wt % with respect with the total fat, such as for example 0 to 10%, 0 to 8%, 0 to 6%, 0 to 4%; or for example 1 to 12%, 2 to 12%. 4 to 12%, 6 to 12%, 8 to 12%, 10 to 12%.

A additional embodiment relates to a synthetic nutritional composition according to the invention, comprising medium-chain fatty acids mainly in the form of triglycerides, in a total amount in the range of 10.1 to 15 wt %, such as 10.5 to 15%, 11 to 15%, 12 to 15%, 13 to 15%, 14 to 15%, or for example 10.1 to 14%, 10.5 to 14%, or 10.5 to 13%, or 11 to 15%, or 12 to 15%, with respect to the total fat, and long chain fatty acids mainly in the form of triglycerides in a total amount in the range of 85 to 89.9 wt % with respect to the total fat, and wherein:
hexanoic acid is in an amount in the range of 0 to 0.5 wt % with respect with the total fat;
octanoic acid is in an amount in the range of 1.55 to 2 wt % with respect with the total fat;
decanoic acid is in an amount in the range of 1 to 1.8 wt % with respect with the total fat; and
dodecanoic acid is in an amount in the range of 8.0 to 12.0 wt % with respect with the total fat.

A yet further embodiment relates to a synthetic nutritional composition according to the invention, comprising medium-chain fatty acids mainly in the form of triglycerides, in a total amount in the range of 10.1 to 15 wt %, such as 10.5 to 15%, 11 to 15%, 12 to 15%, 13 to 15%, 14 to 15%, or for example 10.1 to 14%, 10.5 to 14%, or 10.5 to 13%, or 11 to 15%, or 12 to 15%, with respect to the total fat, and long chain fatty acids mainly in the form of triglycerides in a total amount in the range of 85 to 89.9 wt % with respect to the total fat, and wherein:
hexanoic acid is in an amount in the range of 0.05 to 0.5 wt % with respect with the total fat;
octanoic acid is in an amount in the range of 1.55 to 2 wt % with respect with the total fat;
decanoic acid is in an amount in the range of 1 to 1.8 wt % with respect with the total fat; and
dodecanoic acid is in an amount in the range of 8.0 to 12.0 wt % with respect with the total fat.

### Fatty Acids—LCFAs

The composition of the invention comprises at least one long chain fatty acid, mainly in the form of triglycerides in an amount of at least 15 wt %.

One embodiment relates to where the at least one long chain fatty acid comprises oleic acid in an amount of at least 30 wt % with respect to total fat; such as from 30 to 50%, 30 to 45%, 35 to 45% oleic acid with respect to total fat.

In one embodiment, the at least one long chain fatty acid is in an amount comprised between 75 and 95 wt % with respect to total fat, for example between 75 and 90 wt % with respect to total fat.

### Fatty Acids—PUFAS

### PUFA

In a particularly advantageous embodiment of the invention, the long chain fatty acids mainly in the form of triglycerides in the composition of the invention comprises at least one polyunsaturated fatty acid (PUFA), the PUFA(s) being present in an amount of at least 0.1 wt % with respect to the total fat.

Polyunsaturated fatty acids can be classified in various groups by their chemical structure. Among those PUFAS one may distinguish the omega-3 and omega-6 PUFAs.

Polyunsaturated omega-3 (ω-3 or n-3) fatty acids comprise alpha-linolenic acid (ALA) 18:3, stearidonic acid (SDA) 18:4, eicosatrienoic acid (ETE) 20:3, n-3 eicosatetraenoic acid (ETA) 20:4, eicosapentaenoic acid (EPA) 20:5, n-3 docosapentaenoic acid (DPA) 22:5, and docosahexaenoic acid (DHA) 22:6. In a particularly advantageous embodiment, the PUFAs according to the invention comprise alpha-linolenic acid, which is an essential fatty acid.

Polyunsaturated omega-6 (ω-6 or n-6) fatty acids comprise linoleic acid 18:2, gamma-linolenic acid (GLA) 18:3, n-6 eicosadienoic acid 20:2, dihomo-gamma-linolenic acid (DGLA) 20:3, arachidonic acid (AA or ARA) 20:4, n-6 docosadienoic acid 22:2, and docosapentaenoic acid 22:5. In a particularly advantageous embodiment, the PUFAs according to the invention comprise linoleic acid, which is an essential fatty acid.

In a particularly advantageous embodiment, the composition of the invention further contains at least one PUFA, which is preferably a n-3 and/or a n-6-PUFA (that is to say a n-3-PUFA, a n-6 PUFA or, more preferably, a mixture of n-3 and n-6-PUFA), the PUFA(s) being present in an amount generally of at least 0.8 wt %, preferably at least 1.0 wt %, with respect to the total fatty acids. This leads (taking into consideration that the total fat comprises 90 to 96 wt % of fatty acids) to an amount of at least about 0.8 wt %, preferably at least about 1.0 wt %, with respect to the total fat.

The n-3-PUFA can be a C20 or a C22 n-3 fatty acid. The C20 or C22 n-3 PUFA is advantageously present in an amount of at least 0.4 wt %, with respect to all the fatty acids in the composition. This leads to an amount of at least about 0.4 wt %, with respect to the total fat of the composition. Preferably the n-3 PUFA is docosahexanoic acid (DHA, C22:6).

The n-6 PUFA can be a C20 or a C22 n-6 fatty acid. The C20 or C22 n-6-PUFA is advantageously present in an amount of at least 0.4 wt % of all fatty acids in the composition. This leads to an amount of at least 0.4 wt %, with respect to the total fat of the composition. Preferably the n-6-PUFA is arachidonic acid (ARA, C20:4).

In a particularly advantageous embodiment, the PUFAs comprise at least 0.4 wt % of docosahexaneoic acid and at least 0.4 wt % of arachidonic acid, with respect to the total fatty acids.

The source of -PUFA may be, for example, egg lipids, fungal oil, low EPA fish oil or algal oil. The -PUFA of the composition of the invention may be provided in small amounts of oils containing high quantities of preformed arachidonic acid and docosahexaenoic acid such as fish oils or microbial oils.

The presence of -PUFAs is especially advantageous for several key health benefits such as cognitive benefits, visual and cognitive development, anti-inflammatory properties, as it is known in the art.

There are many reports in the literature suggesting that these fatty acids may be essential for optimal cognitive function development. Among its many roles, DHA influences the function of the blood-brain barrier, the activity of membrane-bound enzymes and ionic channels, dopaminergic and serotoninergic neurotransmission, and signal transduction (Yaboob, P Annu. Rev. Nutr. 2009.29:257-282).

One embodiment relates to the synthetic nutritional composition according to the invention wherein it comprises one or more of the following, the percentages being with respect to the total fat:
Linoleic acid (LA) in an amount in the range of 10 to 25 wt %;
Linolenic acid (ALA) in an amount in the range of 0.5 to 5 wt %;
Docosahexaneoic acid (DHA) in an amount in the range of 0.1-2 wt %
Arachidonic acid (ARA) in an amount in the range of 0.1 to 5 wt %.

In one particular embodiment, the invention relates to a synthetic nutritional according to the invention, wherein the fat consists essentially, of a mixture of 0 to 20 wt % of milk fat; 12 to 25 wt % of high oleic sunflower oil; 15 to 20 wt % of sunflower oil; 20 to 35 wt % of canola oil; 5 to 20 wt % of coconut oil; 0 to 13 wt % of palmitic acid mainly in the form of triglycerides, preferably wherein 0 to 6.5 wt % of the palmitic acid is esterified in the sn 1,3 position of the triglycerides; and 0.5 to 2.5 wt %, for example 1 to 2.5 wt % of a mixture of poly-unsaturated fatty acids ARA and DHA, preferably in a ratio ARA:DHA of about 1:1, with respect to total fat.

### Palmitic Acid

The presence of palmitic acid mainly in the form of triglycerides, when the palmitic acid is esterified in the sn-2 position of the triglycerides, is especially advantageous for growth benefits, as it is known in the art (Innis S M, Adv Nutr 2011 May 2(3):275-283.

However palmitic acid esterified in the sn-1,3 position of the triglycerides is generally present in a high amount in standard infant formula. When sn 1,3 palmitic acid is digested, free fatty acids are released which form complexes with minerals, i.e. soaps. These soaps are not absorbable and are lost in faeces. Thus, it is desired to have lower levels of sn 1,3 palmitic acid.

In the synthetic nutritional composition of the present composition, the total levels of sn 1,3 palmitic acid are reduced, in part by the reduction in total amount of palmitic acid present in the compositions of the invention.

Without wishing to be bound by theory, it is believed that when non-absorbed palmitic acid soaps reach the ileum, they may stimulate the L cells to release the hormone GLP1, which is known to slow down gastric emptying rate. Thus, reducing the amount of palmitic acid soaps may contribute to the increased gastric emptying rate.

The nutritional composition of the invention may comprise palmitic acid, mainly (i.e. at least 98%, such as more than 98.5%, or more than 99% or more than 99.5%) in the form of triglycerides, in an amount in the range of from 0 to 13% with respect to total fat; such as from 0 to 12, 0 to 10, 0 to 9, 1 to 8, 2 to 6, 2 to 5, 2 to 4, 2 to 3.5 wt % palmitic acid.

Specific embodiments relate to where the nutritional composition of the invention comprises 0 to 9% palmitic acid, such as 0 to 8.5%, such as 0.5 to 8.5 wt % palmitic acid.

Further specific embodiments of the nutritional composition of the invention comprises no more than 7 wt % palmitic acid sn 1,3, with respect to total fat. For example, the nutritional composition of the invention may comprise palmitic acid sn 1,3 in an amount no more than 7 wt %, with respect to total fat, such as an amount from 0 to 7 wt %, 1 to 7 wt %, 1 to 6.5 wt %, 1 to 6 wt %; or such as from 2 to 7, 3 to 7, 4 to 7, 5 to 7 or 6 to 7 wt %; or such as from 2.5 to 3.5 wt % or for example 0.1 to 4, 0.1 to 3 wt % with respect to total fat.

A further embodiment relates to nutritional composition of the invention wherein palmitic acid sn-2 represents from 15 to 30 wt % of total palmitic acid and wherein palmitic acid sn-1,3 represents from 70 to 75 wt % of total palmitic acid.

### Coconut Oil

Further embodiments relate to the synthetic nutritional composition according to the invention wherein the composition comprises coconut oil in an amount in the range of 10 to 25% with respect to total fat.

One specific embodiment relates to a synthetic nutritional composition according to the invention comprising coconut oil in an amount in the range of 10 to 25% with respect to total fat, such as for example 10 to 22, 10 to 20, 10 to 18, 10 to 16, 10 to 14, 10 to 12 wt %; or for example 12 to 24, 14 to 24, 16 to 24, 18 to 24, 20 to 24 or 22 to 24 wt %; or for example 16 to 20, 18 to 20 wt % with respect to total fat.

Coconut oil contains a mix of medium chain fatty acids which is close to that found in human milk.

### Fat Blends

One specific embodiment of the invention is wherein the nutritional composition according to the invention is a fat blend, and consists essentially of fat. This fat blend is useful for preparing further nutritional compositions, such as infant formulas, particularly for infants who were born preterm or with low-birth weight or experienced intra-uterine growth retardation or who suffered from growth delays due to disease and/or malnutrition, and/or infants who suffer from or are at risk of gut and/or liver disease.

The fat blend may be the sole source of fat in a synthetic nutritional composition according to the invention, for example an infant formula.

Thus, the invention also relates to the use of the fat blend according to the invention for the preparation of a further synthetic nutritional composition according to the invention, for example in preparation of an infant formula; a fortifier, such as a milk fortifier; a starter formula, a follow-on formula; a growing up milk; or a supplement or complement according to the invention.

### Formulas

The synthetic nutritional compositions according to the invention may further comprise a protein source and/or a carbohydrate source.

The synthetic nutritional compositions according to the invention may be for example an infant formula; a fortifier, such as a milk fortifier; a starter formula, a follow-on formula; a growing up milk; or a supplement or complement. The synthetic nutritional composition of the invention may be in the form of a powder, liquid or concentrated liquid. The synthetic nutritional composition of the invention, e.g. an infant formula, may be a milk-based nutritional composition and may be based on a cow's milk, goat's milk or buffalo milk.

In some advantageous embodiments, the synthetic nutritional composition of the invention is an infant formula. The infant formula may be a starter formula generally for infants that are less than 6 months old or a follow-on formula generally for infants or young children that are more than 6 months old. The composition of the invention may also be a growing up milk, or a fortifier such as a human milk fortifier.

According to a particularly advantageous embodiment, the synthetic nutritional composition according to the invention is specially adapted for a vulnerable infant or young child, such as infants and/or young children with LBW, who are critically ill, who were born preterm, who were small for gestational age, who experienced intra-uterine growth retardation and/or who suffered from growth delays due to disease and/or malnutrition. In further embodiments, the synthetic nutritional composition is specially adapted for infants and/or young children who have been fed parenterally or are at risk for becoming candidates for parenteral feeding.

In one particular embodiment, the synthetic nutritional composition according to the invention is adapted for enteral administration, for example enteral, oro-gastric or nasogastric administration. Administering the formula enterally contributes to stimulating gut maturation, and avoids the risks associated with parenteral feeding, such as line infections, gut atrophy and hepatobiliary disorders.

The quantities of all the components expressed herein as weight % (wt %) with respect to the total fat, reflect the amounts of some components of the fat present in the synthetic nutritional composition, to be consumed by the infant or young child. For example, the composition may be a powdered infant formula that is diluted with water to give a final liquid product. The composition according to the invention may also be a concentrated liquid that is diluted with water to achieve the final liquid product. The composition of the invention may be a liquid product that is directly consumed by the infant as it is. The composition according to the invention may be a human milk fortifier that is added to or diluted with human milk. In this case, the concentration of the components already present in the human milk (to which the human milk fortifier is added) are to be taken as the average values for lactating mothers that are known or predicted from published clinical data.

The composition according to the present invention contains a source of lipids. The lipid source may be any lipid or fat which is suitable for use in infant formulae, as far as the content of MCFAs and LCFAs specific proportions meets the requirements of the invention. Preferred fat sources include palm oleic, high oleic sunflower oil and high oleic sunflower oil. The essential fatty acids linoleic and α-linolenic acid may also be added. In the composition, the fat source (including optional-PUFA such as ARA and/or DHA) preferably has a ratio of n-6 to n-3 fatty acids of about 1:2 to about 10:1, preferably about 5:1 to about 10:1, even more preferably about 7:1 to about 9:1.

In one particularly advantageous embodiment, the composition according to the invention is such that the fat consists essentially of a mixture of 0 to 20% of milk fat; 10 to 30% of coconut oil; 10 to 40% of canola oil; 10 to 25% sunflower oil; 10 to 30% of high oleic sunflower oil; 4 to 10% of palmitic acid mainly in the form of triglycerides; preferably 1.5 to 2.5%, of palmitic acid sn-2 (% of total fat) and 5 to 7% of palmitic acid sn 1,3 of total fat; and 0.5 to 2.5% of a mixture of poly-unsaturated fatty acids ARA and DHA (such as a mixture in 1:1 proportion of ARASCO® and DHASCO® from Martek), preferably in a ratio ARA:DHA of about 1:1, with respect to the total fat.

The milk fat could be replaced efficiently by Betapol®, or by Infat® from AAK/Enzymotec.

Each one of these fat sources may be a refined oil suitable for infant nutrition applications.

Other standard ingredients known to the skilled person for formulating an infant formula, a fortifier or a growing-up milk may also be present in the compositions of the invention.

Thus, the composition of the invention may contain other ingredients which may act to enforce the technical effect of the components, particularly according to the Commission Directive 2006/141/EC of 22 Dec. 2006 on infant formulae and follow-on formulae.

The composition according to the present invention can also contain a carbohydrate source, preferably as prebiotics, or in addition to prebiotics. Any carbohydrate source conventionally found in infant formulae such as lactose, saccharose, maltodextrin, starch and mixtures thereof may be used although the preferred source of carbohydrates is lactose.

The prebiotics that may be used in accordance with the present invention are not particularly limited and include all food substances that promote the growth of probiotics or health beneficial micro-organisms in the intestines. Preferably, they may be selected from the group consisting of oligosaccharides, optionally containing fructose, galactose, and mannose; dietary fibers, in particular soluble fibers, soy fibers; inulin; or mixtures thereof. Some examples of prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), inulin, polydextrose oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof. In a particular embodiment, the prebiotics may be fructooligosaccharides and/or inulin. In a specific embodiment, the prebiotics is a combination of FOS with inulin such as in the product sold by BENEO-Orafti under the trademark Orafti® oligofructose (previously Raftilose®) or in the product sold by BENEO-Orafti under the trademark Orafti® inulin (previously Raftiline®). Another example is a combination of 70% short chain fructo-oligosaccharides and 30% inulin, which is registered by Nestle under the trademark “Prebio 1”.

In one embodiment, the synthetic nutritional composition according to the invention doesn't comprise any prebiotic oligosaccharide.

The prebiotics can also be a BMO (bovine's milk oligosaccharide) and/or a HMO (human milk oligosaccharide) such as N-acetylated oligosaccharides, sialylated oligosaccharides, fucosylated oligosaccharides and any mixtures thereof.

A particular example of prebiotic is a mixture of galacto-oligosaccharide(s), N-acetylated oligosaccharide(s) and sialylated oligosaccharide(s) in which the N-acetylated oligosaccharide(s) represent 0.5 to 4.0% of the oligosaccharide mixture, the galacto-oligosaccharide(s) represent 92.0 to 98.5% of the oligosaccharide mixture and the sialylated oligosaccharide(s) represent 1.0 to 4.0% of the oligosaccharide mixture. This mixture is hereinafter referred to as “CMOS-GOS”. For example a composition for use according to the invention can contain from 2.5 to 15.0 wt % CMOS-GOS on a dry matter basis with the proviso that the composition comprises at least 0.02 wt % of an N-acetylated oligosaccharide, at least 2.0 wt % of a galacto-oligosaccharide and at least 0.04 wt % of a sialylated oligosaccharide. WO2006087391 and WO2012160080 provide some examples of production of CMOS-GOS.

In particular, the human milk oligosaccharides, for example sialylated oligosaccharides, described in WO 2012/069416 published on May 31, 2012 may be included in the composition according to the invention.

Probiotic may be added to the composition according to the invention. All probiotic micro-organisms may be added additionally. Preferably, the probiotic may be selected for this purpose from the group consisting of Bifidobacterium, Lactobacillus, Lactococcus, Enterococcus, Streptococcus, Kluyveromyces, Saccharoymces, Candida, in particular selected from the group consisting of Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof. Some suitable examples of probiotics are selected from the group consisting of Bifidobacterium longum NCC3001 (ATCC BAA-999), Bifidobacterium longum NCC2705 (CNCM I-2618), Bifidobacterium longum NCC490 (CNCM I-2170), Bifidobacterium lactis NCC2818 (CNCM I-3446), Bifidobacterium breve strain A, Lactobacillus paracasei NCC2461 (CNCM I-2116), Lactobacillus johnsonii NCC533 (CNCM I-1225), Lactobacillus rhamnosus GG (ATCC53103), Lactobacillus rhamnosus NCC4007 (CGMCC 1.3724), Enterococcus faecium SF 68 (NCC2768; NCIMB10415), and mixtures thereof.

The composition according to the invention can also contain a protein source. The type of protein is not believed to be critical to the present invention provided that the minimum requirements for essential amino acid content are met and satisfactory growth is ensured. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions. The proteins can be at least partially hydrolyzed in order to enhancement of oral tolerance to allergens, especially food allergens. In that case the composition is a hypoallergenic composition.

In a preferred embodiment, the composition may be cow's milk whey based infant formula. The formula may also be a hypoallergenic (HA) formula in which the cow milk proteins are (partially or extensively) hydrolysed. The formula may also be based on soy milk or a non-allergenic formula, for example one based on free amino acids.

The composition of the invention can also contain all vitamins and minerals, and other micronutrients, understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, the composition of the invention may contain emulsifiers and stabilisers such as soy, lecithin, citric acid esters of mono- and di-glycerides, and the like.

The composition of the invention may also contain other substances which may have a beneficial effect such as lactoferrin, nucleotides, nucleosides, gangliosides, polyamines, and the like.

The preparation of the composition according to the invention will now be described by way of example.

The formula may be prepared in any suitable manner. For example, it may be prepared by blending together a protein source, a carbohydrate source, and a fat source, for example a fat blend according to the invention. If used, the emulsifiers may be included at this point. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture. The liquid mixture is then homogenized, for example in two stages.

The liquid mixture may then be thermally treated to reduce bacterial loads, by rapidly heating the liquid mixture to a temperature in the range between about 80° C. and about 150° C. for duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenized, for example in two stages between about 10 MPa and about 30 MPa in the first stage and between about 2 MPa and about 10 MPa in the second stage. The homogenized mixture may then be further cooled to add any heat sensitive components, such as vitamins and minerals. The pH and solids content of the homogenized mixture are conveniently adjusted at this point.

The homogenized mixture is transferred to a suitable drying apparatus such as a spray dryer or freeze dryer and converted to powder. The powder should have a moisture content of less than about 5% by weight. Some of the carbohydrate may be added at this stage by dry-mixing along with optional probiotic bacterial strain(s), or by blending them in a syrup form of crystals, along with optional probiotic bacterial strain(s), and spray-dry (or freeze-dry).

If a liquid composition is preferred, the homogenized mixture may be sterilized then aseptically filled into suitable containers or may be first filled into the containers and then retorted.

In another embodiment, the composition of the invention may be a supplement in an amount sufficient to achieve the desired effect in an infant. This form of administration is usually more suited to preterm infants.

The supplement may be in the form of powder, tablets, capsules, pastilles or a liquid for example, as long as it is a suitable nutritional composition for the infant. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

The supplement can be added in a product acceptable to the consumer (who is an infant), such as an ingestible carrier or support, respectively. Examples of such carriers or supports are a pharmaceutical or a food composition. Examples for such compositions are infant formula including preterm formula.

Further, the supplement may contain an organic or inorganic carrier material suitable for enteral or parenteral administration as well as vitamins, minerals trace elements and other micronutrients in accordance with the recommendations of Government bodies such as the European Commission Directive 2006/141/EC of 22 Dec. 2006 on infant formulae and follow-on formulae.

### Target Groups

The synthetic nutritional composition of the invention may be advantageously administered to infants and/or young children. In a particular embodiment, it is used for infant of less than 12 months, less than 6 months or less than 3 months or less. In a particular embodiment, the composition is administered to a preterm infant.

In one embodiment of the invention, the composition of the invention is an infant formula (or a follow-on formula or a growing up milk or a human milk fortifier), intended to be administered to a child, such as vulnerable infants and/or young children, such as such as infants and/or young children with LBW, who are critically ill, who were born preterm, who were small for gestational age, who experienced intra-uterine growth retardation, and/or who suffered from growth delays due to disease and/or malnutrition, who have been fed enterally or are at risk for being partially enterally fed or are at risk of becoming candidates for eneteral feeding.

In one embodiment, the synthetic nutritional composition of the invention is designed for consumption by infants from birth to one year old. In some embodiments, it is specifically designed to meet the nutritional needs of preterm infants.

In one embodiment, the synthetic nutritional composition of the invention is designed for consumption by infants or young children who have reduced gut motility, which may for example be due to gut immaturity. For example, preterm infants may suffer from gut immaturity and reduced gut motility.

In one embodiment, the synthetic nutritional composition of the invention is designed for consumption by infants or young children who have poor liver health or are at risk for developing liver disease. In particular, ELBW infants often display liver disease, and therefore in one particular embodiment relates to the synthetic nutritional composition designed for such infants.

In some embodiments of the invention, the synthetic nutritional composition is for use in infants or young children who are sick. In some embodiments it may be use in infants or young children who are critically ill, i.e. with a life threatening illness or injury.

In certain embodiments, the synthetic nutritional composition according to the invention is adapted for oral administration. In other embodiments, the synthetic nutritional composition according to the invention is adapted for intragastric (e.g. oro-gastric, nasogastric), intraduodenal or intrajejunal administration.

The composition can be administered (or given) at an age and for a period that depends on the needs.

For example the composition can be given immediately after birth of the infants. The composition of the invention can also be given during the first week of life of the infant, or during the first 2 weeks of life, or during the first 3 weeks of life, or during the first month of life, or during the first 2 months of life, or during the first 3 months of life, or during the first 4 months of life, or during the first 6 months of life, or during the first 8 months of life, or during the first 10 months of life, or during the first year of life, or during the first two years of life or even more. In some other embodiments, the composition of the invention is given few days, or few weeks, or few months after birth.

In one embodiment the composition of the invention is given to the infant or young child as a supplementary composition to the mother's milk. In one embodiment the composition is given to the infant or young child as the sole or primary nutritional composition during at least one period of time, e.g. after the 1st, 2nd or 4th month, during at least 1, 2, 4 or 6 months. In some embodiments the infant or young child receives the mother's milk during at least the first 2 weeks, first 1, 2, 4, or 6 months. In one embodiment the composition of the invention is given to the infant or young child after such period of mother's nutrition, or is given together with such period of mother's milk nutrition.

In some other embodiments of the present invention, the synthetic nutritional compositions of the present invention are intended for patients regardless of age, especially critically ill patients regardless of age, in particular such patients who have been fed enterally and/or who are at risk for being fed enterally. In specific embodiments, the invention relates to adults, especially critically ill adults. Adults are defined as individuals 13 years or older. Other specific embodiments relate to children, especially critically ill children. Children are defined as individuals older than 3 and younger than 13. Further specific embodiments relate to elderly, especially elderly who are critically ill and/or feeding intolerant. Elderly is defined as age 60 and above. These particular embodiments of the invention need not adhere to the regulations concerning infant formulae.

Uses of Nutritional Composition

Preterm and critically ill infants and young children may suffer from intolerance to enteral feeds. Feeding intolerance prompts health care provides to interrupt enteral feeds and supply nutrition by parenteral nutrition.

Another aspect of the invention relates to a synthetic nutritional composition according to the invention for use in promoting and/or ensuring gut maturation, and/or reducing the risk of gut atrophy in an infant or a young child; and/or reducing the accumulation of gastric residuals in infants or young children; in particular in vulnerable infants and/or young children, such as such as infants and/or young children with LBW, who are critically ill, who were born preterm, who experienced intra-uterine growth retardation, who suffered from growth delays due to disease and/or malnutrition, who were small for gestational age, who have been fed enterally or are being partially enterally fed or are at risk of becoming candidates for enteral feeding. Specific embodiments relate to nutritional composition for use according to the invention wherein the use is in preterm infants, even more specifically in LBW infants. In one embodiment, the infant or young child may be critically ill.

Liver function is immature in preterm infants, and hepatic dysfunction is frequent in preterm infants, affecting up to 50% of extremely low birth weight (ELBW) infants.

Thus, in another embodiment, the invention relates to a synthetic nutritional composition according to the invention for use in promoting and/or ensuring liver maturation, and/or reducing the risk of liver disease in an infant or a young child, such as infants and/or young children with LBW, who are critically ill, who were born preterm, who were small for gestational age, who experienced intra-uterine growth retardation, who suffered from growth delays due to disease and/or malnutrition, who have been fed enterally or are being partially enterally fed or are at risk for becoming candidates for enteral feeding, in particular preterm infants, even more specifically in LBW and/or ELBW preterm infants.

Alternatively, this aspect of the invention may be described as relating to the use of a the synthetic nutritional composition according to the invention, in particular the fat blend according to the invention, in preparing a synthetic nutritional composition according to the invention, in particular in preparing a nutritional composition for promoting and/or ensuring gut maturation, or reducing the risk of gut atrophy in an infant or a young child; and/or reducing the accumulation of gastric residuals in an infant or a young child, and/or reducing the risk of regurgitation and/or vomiting, and or improving tolerance to feeds, in particular in preterm infants, even more specifically in LBW infants.

In another embodiment, the invention relates to a synthetic nutritional composition according to the invention for use in increasing gastric emptying rate, or for improving gastric motility, particularly in in an infant or a young child, in particular preterm infants, even more specifically in LBW preterm infants.

In further embodiments, the invention relates to a synthetic nutritional composition according to the invention for use in reducing the risk of regurgitation and/or vomiting and or improving tolerance to feeds, for example in an infant and/or young child, in particular in preterm infants, even more specifically in LBW and/or ELBW infants.

Preterm infants are at increased risk of insulin resistance, hyperglycemia, poor nutrient utilization, impaired lean body mass growth, fat accumulation in the visceral area and metabolic disease later in life.

Thus, in another embodiment, the invention relates to a synthetic nutritional composition according to the invention for use such as for use in use in promoting insulin sensitivity, improving glucose management, improving nutrient utilization, improving lean body mass growth, reducing visceral adiposity and/or reducing the risk of metabolic disease later in life in an infant or a young child, in particular in a vulnerable infant or young child, such as infants and/or young children with LBW, who were small for gestational age, who are critically ill, who were born preterm, who experienced intra-uterine growth retardation and/or who suffered from growth delays due to disease and/or malnutrition, who have been fed enterally or are at risk for becoming candidates for enteral feeding. In a particular embodiment, the synthetic nutritional composition according to the invention is for use in preterm infants, even more specifically in LBW and/or ELBW preterm infants.

A final aspect relates to a method of treating and/or preventing liver disease, diabetes, obesity and/or metabolic syndrome, comprising administering a synthetic nutritional composition according to the invention to an individual in need thereof.

It should be noted that embodiments and features described in the context of one of the aspects of the present invention also apply to the other aspects of the invention.

In particular it should be noted that embodiments and features described in the context of infant and young children may also apply to other groups, such as children, adults, elderly individuals and/or critically ill children, adults or elderly individuals.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

### EXAMPLES

### Example 1

A cross-over study was designed where each subject received all test meals. Subjects were randomly assigned to one of 10 possible randomized sequences of the 5 test products. Each subject received the 5 study products during 5 different visits, separated by at least 1-week washout, and at most 4 weeks. Each visit spread over two days (D−1 and D0):
Day −1 for getting standardized meals
Day 0 for:
   Baseline plasma samples
   Study product intake
   Plasma samples-Breath samples

The subjects were healthy males from 20 to 40 years old with a BMI of 19 to 24.9 kg m⁻², and which had a normal fasting glycemia.

### Study Description

The five study products contained the same amount of maltodextrins and whey protein, but differed in their fatty acid composition. All study products contain a third of each subject's daily energy requirement, as determined by the Harris Benedict equation multiplied by a factor 1.5 to account for physical activity.
1. Maltodextrin (43% TEI)+whey proteins (WP, 12% TEI)
2. Maltodextrin (43% TEI)+WP (12% TEI)+MUFA (39% TEI)+PUFA (6% TEI)
3. Maltodextrin (43% TEI)+WP (12% TEI)+MUFA (27% TEI)+MCT (12% TEI)+PUFA (6% TEI)
4. Maltodextrin (43% TEI)+WP (12% TEI)+MUFA (12% TEI)+MCT (27% TEI)+PUFA (6% TEI)
5. Maltodextrin (43% TEI)+WP (12% TEI)+MCT(39% TEI)+PUFA (6% TEI).

(TEI=total energy intake; WP=whey proteins; MUFA=Monounstaturated fatty acids; PUFA=Polyunsaturated fatty acids; MCT=medium chain triglycerides) See also Table 1 for composition of meals.

100 mg of 13C-octanoate (Eurisotope, St Aubin, France) was added to each study product to monitor gastric emptying.

TABLE 1
Fatty acid profile of study products

| | Meal 2 | Meal 3 | Meal 4 | Meal 5 |
|---|---|---|---|---|
| Total Fatty acid methyl esters | 90.24 | 91.53 | 85.21 | 87.30 |
| (FAME) (g/100 g fat) | | | | |
| Saturates (g/100 g product) | 0.90 | 2.50 | 6.4 | 8.10 |
| Mono unsaturates (g/100 g | 8.60 | 7.40 | 2 | 1.30 |
| product) | | | | |
| Poly unsaturates (g/100 g | 1.60 | 1.80 | 1.4 | 1.30 |
| product) | | | | |
| (g/100 g fat) | | | | |
| C6:0, Hexanoic | 0.00 | 0.00 | 0.00 | 0.00 |
| C8:0, Octanoic | 0.00 | 7.31 | 29.04 | 35.53 |
| C10:0, Decanoic | 0.00 | 5.57 | 22.34 | 27.07 |
| C12:0, Dodecanoic | 0.00 | 0.09 | 0.21 | 0.18 |
| C14:0, Myristic | 0.06 | 0.07 | 0.07 | 0.05 |
| C16:0, Palmitic | 3.61 | 3.34 | 2.33 | 2.11 |
| C16:1 n-7, Palmitoleic | 0.12 | 0.12 | 0.05 | 0.00 |
| C18:0, Stearic | 2.51 | 2.13 | 1.08 | 1.04 |
| C18:1 n-9, Oleic | 69.13 | 57.18 | 17.11 | 10.24 |
| C18:2 n-6, Linoleic | 10.49 | 10.67 | 10.15 | 9.40 |
| C18:3 n-3, alpha-Linoleic | 2.26 | 3.20 | 2 | 1.07 |
| C20:0, Arachidic | 0.31 | 0.31 | 0.14 | 0.09 |
| C20:1 n-9, Eicosenoic | 0.51 | 0.57 | 0.25 | 0.06 |
| Total MCT | 0.00 | 12.97 | 51.59 | 62.78 |
| Total essential FA | 12.75 | 13.87 | 12.15 | 10.47 |
| Total LCFA | 88.94 | 77.52 | 33.11 | 24.01 |

Meals are per-subject isocaloric, except for M1 which is the fat-free reference diet and has similar carbohydrate and protein content to the other meals, but no lipid. All other meals one third of each subject's daily energy requirement, M2 and M3 are dominated by oleic acid, M4 and M5 by MCT.

TABLE 2
Composition of Meal 1 (M1)

| | | | | |
|---|---|---|---|---|
| | Lipid | | | Amount |
| | composition | | Amount | per |
| | (% final oil | % of | per diet | 100 g |
| Meal 1 | mix) | TEI | (g) | (m/m) |

| | | | |
|---|---|---|---|
| maltodextrin (≈3 wt % water) | 43 | 107.50 | 24.57 |
| Whey protein | 12 | 30.00 | 6.86 |
| Water to add | | 300.00 | 68.57 |
| Total without additives (kg) | | 437.50 | 100.00 |
| TS final beverage (% m/m) | | | 31% |
| Masking LG | | | 0.19 |
| Stevia | | | 0.01 |
| Strawberry aroma | | | 0.08 |

TABLE 3
Composition of Meal 2 (M2)

| | Lipid | | | |
|---|---|---|---|---|
| | composition | | Amount | Amount |
| | (% final oil | % of | per | per 100 g |
| Meal 2 | mix) | TEI | diet (g) | (m/m) |
| maltodextrin | | 43 | 107.50 | 24.57 |
| Whey protein | | 12 | 30.00 | 6.86 |
| MCT 40% emulsion incl. | | | 0.00 | 0.00 |
| Citrem | | | | |
| lipids | 0 | 0 | 0.00 | 0.00 |
| emulsion water | | | 0.00 | 0.00 |
| Soybean oil 40% emulsion | | | 0.00 | 0.00 |
| incl. Citrem | | | | |
| lipids | 0 | 0 | 0.00 | 0.00 |
| emulsion water | | | 0.00 | 0.00 |
| Canola oil 40% emulsion | | | 37.50 | 8.57 |
| incl. Citrem | | | | |
| lipids | 30 | 14 | 15.00 | 3.43 |
| emulsion water | | | 21.00 | 4.80 |
| High oleic sunflower oil 40% | | | 87.50 | 20.00 |
| emulsion incl. Citrem | | | | |
| lipids | 70 | 32 | 35.00 | 8.00 |
| emulsion water | | | 49.00 | 11.20 |
| Total Citrem (4% m/m | | | 5.00 | 1.14 |
| emulsion) | | | | |
| Water to add | | | 175.00 | 40.00 |
| Total without additives | | | 437.50 | 100.00 |
| (kg) | | | | |
| TS final beverage (% m/m) | | | | 43% |
| Masking LG | | | | 0.19 |
| Stevie | | | | 0.018 |
| Strawberry aroma | | | | 0.08 |

TABLE 4
Composition of Meal 3 (M3)

| | Lipid | | | Amt |
|---|---|---|---|---|
| | composition | | Amount | per |
| | (% final | % of | per diet | 100 g |
| Meal 3 | oil mix) | TEI | (g) | (m/m) |
| maltodextrin | | 43 | 107.50 | 24.57 |
| Whey protein | | 12 | 30.00 | 6.86 |
| MCT 40% emulsion incl. Citrem | | | 20.00 | 4.57 |
| lipids | 16 | 7.2 | 8.00 | 1.83 |
| emulsion water | | | 11.20 | 2.56 |
| Soybean oil 40% emulsion incl. | | | 0.00 | 0.00 |
| Citrem | | | | |
| lipids | 0 | 0 | 0.00 | 0.00 |
| emulsion water | | | 0.00 | 0.00 |
| Canola oil 40% emulsion incl. | | | 52.50 | 12.00 |
| Citrem | | | | |
| lipids | 42 | 18.9 | 21.00 | 4.80 |
| emulsion water | | | 29.40 | 6.72 |
| High oleic sunflower oil 40% | | | 52.50 | 12.00 |
| emulsion incl. Citrem | | | | |
| lipids | 42 | 19 | 21.00 | 4.80 |
| emulsion water | | | 29.40 | 6.72 |
| Total Citrem (4% m/m | | | 5.00 | 1.14 |
| emulsion) | | | | |
| Water to add | | | 175.00 | 40.00 |
| Total without additives (kg) | | | 437.50 | 100.00 |
| TS final beverage (% m/m) | | | | 43% |
| Masking LG | | | | 0.19 |
| Stevie | | | | 0.01 |
| Strawberry aroma | | | | 0.08 |

TABLE 5
Composition of Meal 4 (M4)

| | Lipid | | | Amount |
|---|---|---|---|---|
| | composition | | Amount | per |
| | (% final oil | % of | per diet | 100 g |
| Meal 4 | mix) | TEI | (g) | (m/m) |
| maltodextrin | | 43 | 107.50 | 24.57 |
| Whey protein | | 12 | 30.00 | 6.86 |
| MCT 40% emulsion incl. Citrem | | | 83.75 | 19.14 |
| lipids | 67 | 30.15 | 33.50 | 7.66 |
| emulsion water | | | 46.90 | 10.72 |
| Soybean oil 40% emulsion incl. | | | 22.50 | 5.14 |
| Citrem | | | | |
| lipids | 18 | 8.1 | 9.00 | 2.06 |
| emulsion water | | | 12.60 | 2.88 |
| Canola oil 40% emulsion incl. | | | 18.75 | 4.29 |
| Citrem | | | | |
| lipids | 15 | 6.75 | 7.50 | 1.71 |
| emulsion water | | | 10.50 | 2.40 |
| High oleic sunflower oil 40% | | | 0.00 | 0.00 |
| emulsion incl. Citrem | | | | |
| lipids | 0 | 0 | 0.00 | 0.00 |
| emulsion water | | | 0.00 | 0.00 |
| Total Citrem (4% m/m | | | 5.00 | 1.14 |
| emulsion) | | | | |
| Water to add | | | 175.00 | 40.00 |
| Total without additives (kg) | | | 437.50 | 100.00 |
| TS final beverage (% m/m) | | | | 43% |
| Masking LG | | | | 0.19 |
| Stevie | | | | 0.01 |
| Strawberry aroma | | | | 0.08 |

TABLE 6
Composition of Meal 5 (M5)

| | Lipid | | | Amount |
|---|---|---|---|---|
| | composition | | Amount | per |
| | (% final oil | % of | per diet | 100 g |
| Meal 5 | mix) | TEI | (g) | (m/m) |
| maltodextrin | | 43 | 107.50 | 24.57 |
| Whey protein | | 12 | 30.00 | 6.86 |
| MCT 40% emulsion incl. Citrem | | | 97.50 | 22.29 |
| lipids | 78 | 35.1 | 39.00 | 8.91 |
| emulsion water | | | 54.60 | 12.48 |
| Soybean oil 40% emulsion incl. | | | 27.50 | 6.29 |
| Citrem | | | | |
| lipids | 22 | 10 | 11.00 | 2.51 |
| emulsion water | | | 15.40 | 3.52 |
| Canola oil 40% emulsion incl. | | | 0.00 | 0.00 |
| Citrem | | | | |
| lipids | 0 | 0 | 0.00 | 0.00 |
| emulsion water | | | 0.00 | 0.00 |
| High oleic sunflower oil 40% | | | 0.00 | 0.00 |
| emulsion incl. Citrem | | | | |
| lipids | 0 | 0 | 0.00 | 0.00 |
| emulsion water | | | 0.00 | 0.00 |
| Total Citrem (4% m/m | | | 5.00 | 1.14 |
| emulsion) | | | | |
| Water to add | | | 175.00 | 40.00 |
| Total without additives (kg) | | | 437.50 | 100.00 |
| TS final beverage (% m/m) | | | | 43% |
| Masking LG | | | | 0.19 |
| Stevie | | | | 0.01 |
| Strawberry aroma | | | | 0.08 |

### Study

On the day of the kinetic, the participants came fasted. There were no other diet restrictions.

The beverage was served at room temperature in an opaque cup with a cover to be consumed orally within 5-10 minutes.

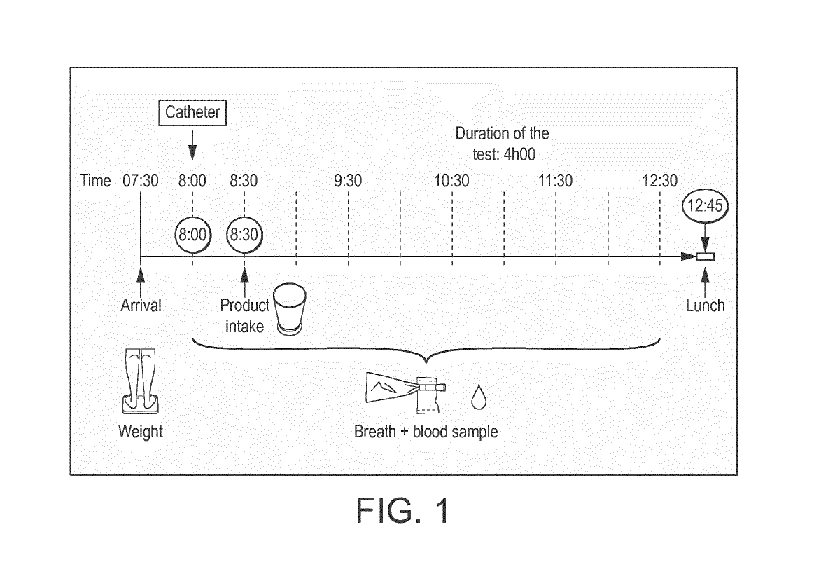

Blood samples were drawn time points starting from 1 hr before intake of the beverage, and during the 4 hours of the study (See further FIG. 1 for overview of study).

### Gastric Emptying

In brief, 100 mg of ¹³C-octanoate was dissolved into the test meals on the evening preceding the test to ensure complete homogeneous dissolution of the tracer. On the day of the metabolic investigation, two baseline breath samples were collected in 3 glass vacutainers at times −30 and −15 min prior to the meal ingestion. All test meals were ingested in less than 10 minutes, and time zero was defined as the end of food ingestion. Breath samples were collected at times 15, 30, 45, 60, 75, 90, 105, 120, 150, 180, 210 and 240 minutes using Easy Sampler Device and Crimp Tubes from QuinTron (Milwaukee, Wis., USA)

Breath ¹³CO₂ samples were analyzed for ¹³C enrichment using an isotope ratio mass spectrometer (IRMS, Delta V Advantage, Thermo, Bremen, Germany) device hyphenated to a gas chromatography system (GC Trace, Thermo, Bremen, Germany). The analyses were performed to assess the ¹³CO₂/¹²CO₂ ratio (expressed as δ¹³C, % o). CO₂ standard gas was calibrated against international standard allowing ¹³C/¹²C ratio to be quoted against the Vienna Pee De Belemnite (VPDB).

Data were transformed using non linear equations according to Ghoos et al (2) using the R software to calculate non-linear curve fitting. The parameter T50Meas is derived from these equations and represents the time in minutes when half of the stomach's content has been emptied.

FIG. 2 shows the half gastric emptying time (T50Meas) results for the different meals studied. As expected, the meal more rapidly emptyied from the stomach was M1, as it did not contain any fat. Surprisingly, within the fat-containing meals M2 and M3, i.e. those meals which have no or low MCT oil, had a faster gastric emptying than M4 and M5.

### FGF-19

FGF-19 (fibroblast growth factor 19) is an intestinal hormone which can reduce the risk of hepatic steatosis and cholestasis, and promote liver growth. This hormone can alsoexert beneficial effects on glucose metabolism by increasing insulin sensitivity and inducing glycogen synthesis. Furthermore, FGF-19 can also promote protein synthesis, and lead to growth of lean body mass (Schaap, 2012; Reue, 2014).

FGF19 was measured by ELISA using commercial kit from R&D Systems.

The results show FGF-19 is more stimulated in M3 and M2, i.e. those meals which are have low or no MCT oil content induce significantly higher levels of FGF_19 as compared to control. The meals M4 and M5 were not significantly different from control.

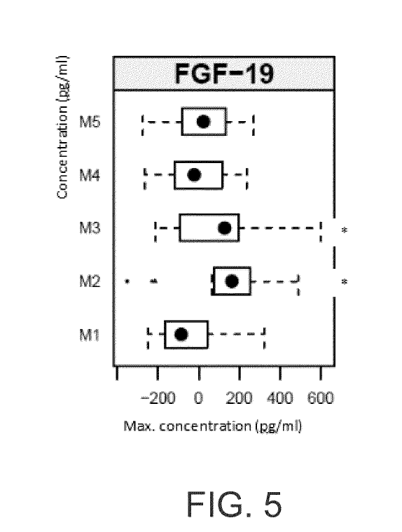

See also FIGS. 3 and 5.

### CCK and Bile Acids

Cholecystokinin (CCK) is an intestinal hormone key to the digestive process as it induces the secretion of the bile acids and pancreatic enzymes required for the digestion of a meal. This hormone also appears to stimulate the maturation of the exocrine pancreas.

CCK was measured by RIA by using a commercial kit from Ray Biotech. Total bile acids were measured by a colorimetric enzymatic assay by using a commercial kit from Bio Supply.

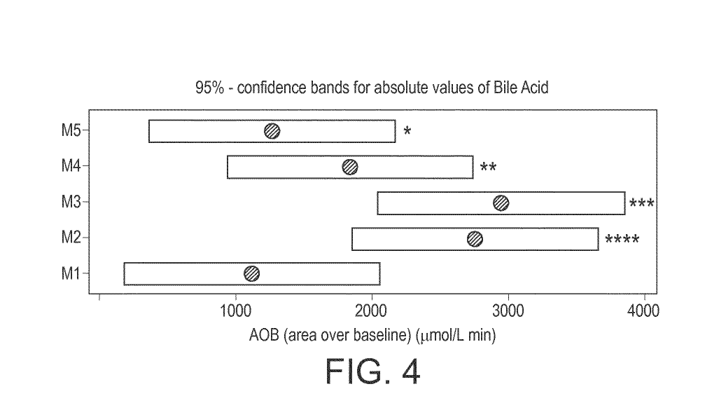

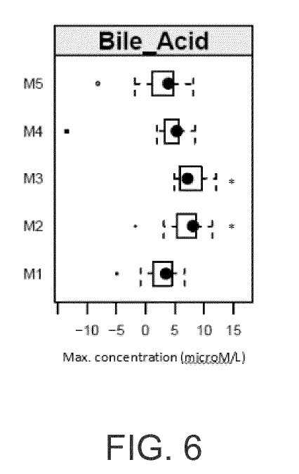

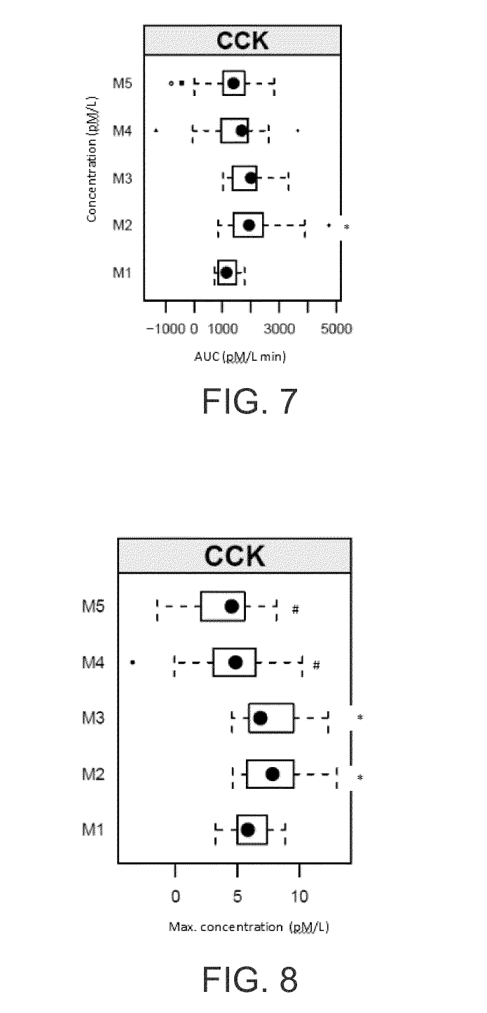

The results show that CCK is more stimulated in M3 and M2, i.e. those meals which are have low or no MCT oil content induce significantly higher levels of CCK as compared to control. By contrast, the meals M4 and M5 induced a lower secretion of CCK as compared to control. Accordingly, bile acids levels were also significantly increased by the M2 and M3 meals only. See also FIGS. 4, 6 and and 7,8.

### Creaming Experiments

Experiments were performed to validate the gastric emptying method.

Method: Samples of each test product (M1-M5) were mixed in equal proportion with simulated gastric juice, which consisted of 2 mg/mL NaCl and 4.5 mg/mL pepsin at 37° C. and a pH of 1.9. The physical distribution of fat throughout each mixture was assessed by measuring optical turbidity as a function of sample height (every 30 um) over time at 37° C. using a Turbiscan™ LAB. Meal 3 was diluted to test stability (Meal 3 (−10%)).

The results are presented below in Table 7.

TABLE 7
In vitro meal coagulation test results

| | | | Cream layer height |
|---|---|---|---|
| | Mixing ratio | | (% of total sample |
| Meal | (meal to SGF) | Creaming rate | height) |
| Meal 2 | 1:1 | ~50 um min−1 | 96.5% |
| Meal 3 | 1:1 | ~1000 um min−1 | 61.9% |
| Meal 3 | 9:1 | ~10 um min−1 | 100% |
| (−10%) | | | |
| Meal 4 | 1:1 | ~300 um min−1 | 74% |
| Meal 5 | 1:1 | ~50 um min−1 | 93.3% |

Conclusions: Meals 2 and 5 displayed no phase separation. Meals 3 and 4 displayed modest creaming, which was due to fat particle aggregation. Such separation in the stomach will be relatively minor and have only a modest effect on gastric emptying. No effect on gastric empting is anticipated for Meals 2, 4, 5.

### Example 2A

TABLE 8
Example of a lipid blend according to the invention

| Ingredients | Variant 1: No Milk Fat |
|---|---|
| MCT Oil | — |
| Palm Olein | — |
| Milk Fat | — |
| Betapol-55 | 15.00 |
| Coconut Oil | 20.00 |
| Canola Oil | 32.21 |
| Sunflower Oil | 16.17 |
| High Oleic Sunflower Oil | 14.38 |
| ARASCO/DHASCO 1:1 | 1.00 |
| ARASCO/DHASCO 2:1 | 1.25 |
| Palmitic Acid (% of total fat) | 8.10 |
| Palmitic Acid sn-2 (% of palmitic | 27.30 |
| acid) | |
| Palmitic Acid sn-1,3 (% of | 72.70 |
| palmitic acid) | |
| Palmitic Acid sn-2 (% of total | 2.20 |
| fat) | |
| Palmitic Acid sn-1,3 (% of total | 5.90 |
| fat) | |

### Example 2B

TABLE 9
Example of lipid blend according to the invention

| Ingredients | Variant 2: No Betapol-55 |
|---|---|
| MCT Oil | — |
| Palm Olein | — |
| Milk Fat | 15.00 |
| Betapol-55 | 0.00 |
| Coconut Oil | 20.00 |
| Canola Oil | 22.87 |
| Sunflower Oil | 18.28 |
| High Oleic Sunflower Oil | 21.59 |
| ARASCO/DHASCO 1:1 | 1.00 |
| ARASCO/DHASCO 2:1 | 1.25 |
| Palmitic Acid (% of total fat) | 7.80 |
| Palmitic Acid sn-2 (% of palmitic | 20.30 |
| acid) | |
| Palmitic Acid sn-1,3 (% of | 79.70 |
| palmitic acid) | |
| Palmitic Acid sn-2 (% of total | 1.60 |
| fat) | |
| Palmitic Acid sn-1,3 (% of total | 6.20 |
| fat) | |

### Example 3

An example of the composition of an infant formula according to the invention is given in the below Table 11.

TABLE 11
example of an infant formula according to the invention

| Nutrient | per 100 kcal | per litre |
|---|---|---|
| Energy (kcal) | 100 | 670 |
| Protein (g) | 1.83 | 12.3 |
| Fat (g) = fat blend of example | 5.3 | 35.7 |
| 2 a) or of example 2 b) | | |
| Linoleic acid (g) | 0.79 | 5.3 |
| α-linoleic acid (mg) | 101 | 675 |
| Lactose (g) | 11.2 | 74.7 |
| Minerals (g) | 0.37 | 2.5 |
| Na (mg) | 23 | 150 |
| K (mg) | 89 | 590 |
| Cl (mg) | 64 | 430 |
| Ca (mg) | 62 | 410 |
| P (mg) | 31 | 210 |
| Mg (mg) | 7 | 50 |
| Mn (μg) | 8 | 50 |
| Se (μg) | 2 | 13 |
| Vitamin A (μg RE) | 105 | 700 |
| Vitamin D (μg RE) | 1.5 | 10 |
| Vitamin E (mg TE) | 0.8 | 5.4 |
| Vitamin K1 (μg) | 8 | 54 |
| Vitamin C (mg) | 10 | 67 |
| Vitamin B1 (mg) | 0.07 | 0.47 |
| Vitamin B2 (mg) | 0.15 | 1.0 |
| Niacin (mg) | 1 | 6.7 |
| Vitamin B6 (mg) | 0.075 | 0.50 |
| Folic acid (μg) | 9 | 60 |
| Pantothenic acid (mg) | 0.45 | 3 |
| Vitamin B12 ((μg) | 0.3 | 2 |
| Biotin (μg) | 2.2 | 15 |
| Choline (mg) | 10 | 67 |
| Fe (mg) | 1.2 | 8 |
| I (μg) | 15 | 100 |
| Cu (mg) | 0.06 | 0.4 |
| Zn (mg) | 0.75 | 5 |

1. A synthetic nutritional composition comprising:
medium-chain fatty acids in a total amount in the range of 5 to 20 wt % with respect to the total fat;
hexanoic acid in an amount in the range of 0 to 0.5 wt % with respect with the total fat;
octanoic acid in an amount in the range of 1.55 to 2 wt % with respect with the total fat;
decanoic acid is an amount in the range of 0 to 1.8 wt % with respect with the total fat;
dodecanoic acid in an amount in the range of 0 to 12.0 wt % with respect with the total fat, and
at least one long chain fatty acid in an amount of at least 15 wt % with respect to the total fat.

2. A synthetic nutritional composition according to claim 1 comprising hexanoic acid in an amount in the range of 0.05 to 0.5 wt % with respect with the total fat.

3. A synthetic nutritional composition according to claim 1 comprising medium-chain fatty acids in a total amount in the range of 10.1 to 15 wt %, with respect to the total fat, wherein:
hexanoic acid is in an amount in the range of 0.05 to 0.5 wt % with respect with the total fat;
octanoic acid is in an amount in the range of 1.55 to 2 wt % with respect with the total fat;
decanoic acid is in an amount in the range of 1 to 1.8 wt % with respect with the total fat;
dodecanoic acid is in an amount in the range of 8.0 to 12.0 wt % with respect with the total fat; and
at least one long chain fatty acid in an amount of at least 15 wt % with respect to the total fat.

4. A synthetic nutritional composition according to claim 1, comprising coconut oil in an amount in the range of 5 to 25 wt % with respect to total fat.

5. The synthetic nutritional composition according to claim 1, comprising, palmitic acid in an amount from 0 to 13 wt %, with respect to total fat.

6. The synthetic nutritional composition according to claim 1, wherein the composition comprises palmitic acid sn 1,3 in an amount of no more than 7 wt % with respect to total fat.

7. The synthetic nutritional composition according to claim 1, wherein the at least one long chain fatty acid comprises oleic acid in an amount of at least 30 wt % with respect to total fat.

8. The synthetic nutritional composition according to claim 1, comprising at least one polyunsaturated fatty acid (PUFA), the PUFA(s) being present in an amount of at least 0.1 wt % with respect to the total fat.

9. The synthetic nutritional composition according to claim 8, comprising one or more of the following, the percentages being with respect to the total fat:
Linoleic acid (LA) in an amount in the range of 10 to 25 wt %;
Linolenic acid (ALA) in an amount in the range of 0.5 to 5 wt %;
Docosahexaneoic acid (DHA) in an amount in the range of 0.1-2 wt %; and
Arachidonic acid (ARA) in an amount in the range of 0.1 to 5 wt %.

10. The synthetic nutritional composition according to claim 1, wherein the fat consists of: a mixture of 0 to 20 wt % of milk fat; 12 to 25 wt % of high oleic sunflower oil; 15 to 20 wt % of sunflower oil; 20 to 35 wt % of canola oil; 5 to 20 wt % of coconut oil; 0 to 13 wt % of palmitic acid; and 0.5 to 2.5 wt % of a mixture of poly-unsaturated fatty acids ARA and DHA with respect to total fat.

11. The synthetic nutritional composition according to claim 1, which is a fat blend and consists essentially of fat.

12. The synthetic nutritional composition according to claim 1, comprising a protein source and/or a carbohydrate source.

13. The synthetic nutritional composition according to claim 12, wherein the nutritional composition is selected from the group consisting of an infant formula, a fortifier, a starter formula, a follow-on formula, a growing up milk, and a supplement or complement.

14. The synthetic nutritional composition according to claim 13, wherein the composition is specially adapted for infants and/or young children.

15. (canceled)

16. A method for ensuring gut maturation, reducing the risk of gut atrophy and/or reducing the accumulation of gastric residuals and/or reducing the risk of regurgitation and/or vomiting and or improving tolerance to feeds, in an infant or young child comprising administering a synthetic nutritional composition comprising: medium-chain fatty acids in a total amount in the range of 5 to 20 wt % with respect to the total fat; hexanoic acid in an amount in the range of 0 to 0.5 wt % with respect with the total fat; octanoic acid in an amount in the range of 1.55 to 2 wt % with respect with the total fat; decanoic acid is an amount in the range of 0 to 1.8 wt % with respect with the total fat; dodecanoic acid in an amount in the range of 0 to 12.0 wt % with respect with the total fat, and at least one long chain fatty acid in an amount of at least 15 wt % with respect to the total fat to an individual in need of same.

17. The method of claim 16 for use in promoting and/or ensuring liver maturation, and/or reducing the risk of liver disease in an infant or a young child.

18. The method of claim 16 for use in promoting insulin sensitivity, improving glucose management, improving nutrient utilization, improving lean body mass growth, reducing visceral adiposity and/or reducing the risk of metabolic disease later in life in an infant or a young child.

19. The method of claim 16 for use in increasing the gastric emptying rate in an infant or young child, in particular in preterm infants, even more specifically in LBW infants.

20. A method of treating and/or preventing liver disease, diabetes, obesity and/or metabolic syndrome, comprising administering a synthetic nutritional composition comprising: medium-chain fatty acids in a total amount in the range of 5 to 20 wt % with respect to the total fat; hexanoic acid in an amount in the range of 0 to 0.5 wt % with respect with the total fat; octanoic acid in an amount in the range of 1.55 to 2 wt % with respect with the total fat; decanoic acid is an amount in the range of 0 to 1.8 wt % with respect with the total fat; dodecanoic acid in an amount in the range of 0 to 12.0 wt % with respect with the total fat, and at least one long chain fatty acid in an amount of at least 15 wt % with respect to the total fat to an individual in need thereof.
